(19) United States
(12) Patent Application Publication
(10) Pub. No.: US 2017/0035273 A1
(43) Pub. Date: Feb. 9, 2017

(54) CONDUIT OPENING/CLOSING APPARATUS AND INSERTION SYSTEM INCLUDING CONDUIT OPENING/CLOSING APPARATUS

(71) Applicant: OLYMPUS CORPORATION, Tokyo (JP)

(72) Inventor: Kahori YASUNAGA, Hachioji-shi (JP)

(73) Assignee: OLYMPUS CORPORATION, Tokyo (JP)

(21) Appl. No.: 15/296,116

(22) Filed: Oct. 18, 2016

Related U.S. Application Data
(63) Continuation of application No. PCT/JP2015/069671, filed on Jul. 8, 2015.

(30) Foreign Application Priority Data
Jul. 30, 2014 (JP) 2014-155536

(51) Int. Cl. A61B 1/00; A61B 1/015

(52) U.S. Cl. CPC A61B 1/00068; A61B 1/015; A61B 1/00119; A61B 1/00128

(57) ABSTRACT

In the conduit opening/closing operation, when a conduit opening/closing section having a configuration where an operation lever is protruded to the vicinity of an operating section is disposed to the operating section, the operator him/herself can operate the operation lever without losing his/her hold of the operating section.

### CROSS-REFERENCE TO RELATED APPLICATIONS

This is a Continuation Application of PCT Application No. PCT/JP2015/069671, filed Jul. 8, 2015, which was published under PCT Article 21(2) in Japanese.

This application is based upon and claims the benefit of priority from prior the Japanese Patent Application No. 2014-155536, filed Jul. 30, 2014 the entire contents of which are incorporated herein by references.

### BACKGROUND OF THE INVENTION

1. Field of the Invention

The present invention relates to a conduit opening/closing apparatus which is disposed to an inserting section of an endoscope and switches conduits at the time of suction, and an insertion system including the conduit opening/closing apparatus.

2. Description of the Related Art

When a treatment is given by a treatment tool of an endoscope apparatus inserted into a body, a suction object such as a biological fluid generated at the time of the treatment, a cleaning liquid, or a removed biotissue is sucked into a suction tube provided to a sheath or the like through a universal cord by using a channel in an inserting section, and accommodated in a predetermined container.

However, in a generated suction object, when an amount of bleeding is large or a clot of blood is large, normal suction cannot cope with an insufficient amount of suction to a production amount or occurrence of clogging of a suction flow path. Thus, a pump is additionally connected to a forceps plug mouthpiece (a forceps port), and suction and discharge are performed by a manual valve operation through a forceps channel. For example, in Patent Literature 1: Jpn. Pat. Appln. KOKAI Publication No. Hei 11-155807, a sheath and a conduit switching valve mechanism which are aligned in an inserting section (a forceps insertion adapter with a switching valve) are proposed. This forceps insertion adapter with the switching valve is disposed to a forceps plug mouthpiece portion provided on a proximal end side of an inserting section, the switching valve is switched by a manual lever operation, suction is performed through a channel, and discharge to the outside of a biological body is carried out.

### BRIEF SUMMARY OF THE INVENTION

According to an embodiment of the present invention, there is provided a conduit opening/closing apparatus comprising: a connecting section which is detachably connected to a fluid flow mouthpiece provided between a grip section to be griped, which is provided on a proximal end side of an inserting section which has a distal end and a proximal end and whose distal end side is inserted into a lumen, and the proximal end of the inserting section; a first pipe-like section which is consecutively connected to the connecting section and provided along an axial direction of the fluid flow mouthpiece; a second pipe-like section which is connected to the first pipe-like section, has a flow path which extends in a direction crossing an axial direction of the first pipe-like section and in which a fluid having passed through the fluid flow mouthpiece flows; a switching valve which is provided in the middle of the flow path, and switches a passing state and a non-passing state of the fluid by connecting or interrupting the flow path on the basis of a turning motion; and an operation lever which is erected on the switching valve, turns the switching valve, and is disposed to be placed on the grip section side of the fluid flow mouthpiece.

Further, according to an embodiment of the present invention, there is provided an insertion system comprising: an inserting section which has a distal end and a proximal end, and whose distal end side is inserted into a lumen; a grip section which is provided on the proximal end side of the inserting section, and gripped by an operator; a fluid flow mouthpiece which is provided between the proximal end of the inserting section and the grip section; a fluid transmitting/receiving conduit which is consecutively connected from an opening portion provided at the distal end of the inserting section to the fluid flow mouthpiece; and the conduit opening/closing apparatus according to claim 1 which is connected to the fluid flow mouthpiece.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing an appearance structure of an endoscope apparatus having an inserting section to which a conduit opening/closing apparatus is disposed according to a first embodiment;

FIG. 2 is a view showing an appearance structural example of the conduit opening/closing apparatus according to the first embodiment;

FIG. 3 is an exploded view showing a structural example of a switching section main body;

FIG. 4 is a view showing an appearance structural example of a conduit opening/closing apparatus according to a second embodiment;

FIG. 5 is a view showing an appearance structural example of a conduit opening/closing apparatus according to a third embodiment;

FIG. 6 is a view showing an appearance structural example of a conduit opening/closing apparatus according to a fourth embodiment;

FIG. 7 is a view showing an appearance structural example of a movable range limiting section of a conduit opening/closing apparatus according to a fifth embodiment;

FIG. 8A is a view showing an appearance structural example of a movable range limiting section of a conduit opening/closing apparatus according to a sixth embodiment; and

FIG. 8B is a view showing a cross-sectional structure showing the conduit opening/closing apparatus from above.

### DETAILED DESCRIPTION OF THE PREFERRED EMBODIMENT

Embodiments according to the present invention will now be described in detail hereinafter with reference to the drawings.

### First Embodiment

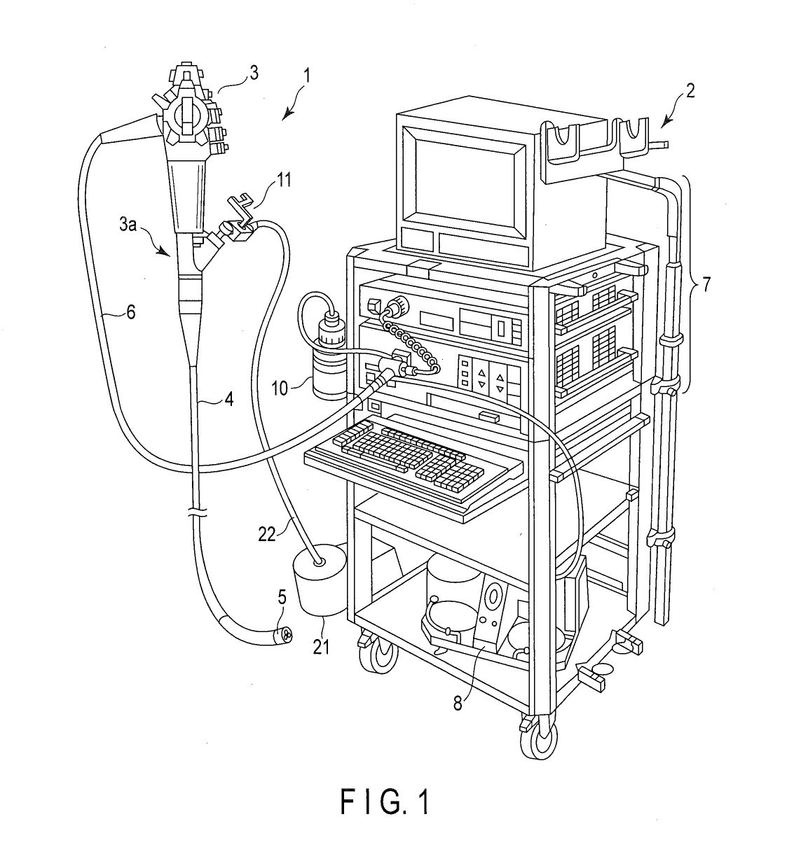

FIG. 1 is a view showing an appearance structure of an endoscope apparatus (an insertion system) having an inserting section to which a conduit opening/closing apparatus is disposed according to a first embodiment.

The endoscope apparatus according to this embodiment is roughly constituted of an endoscope main body (an insertion device) 1 including an inserting section 4, and an endoscope component unit 7 mounted on a movable trolley 2. This embodiment will be described while taking a flexible scope to observe a lumen such as a body cavity of a living body as an example, and it can be likewise mounted in a rigid scope.

The endoscope main body 1 is constituted of the inserting section (a flexible tube) 4 which has a distal end and a proximal end and whose distal end side is inserted into a lumen as an observation target, a bending section 5 which is provided at the distal end of the inserting section 4, and an operating unit 3 which is provided on the proximal end side of the inserting section 4 and operates various functions of the inserting section 4 to bend the bending section 5. At the distal end of the inserting section 4, i.e., on a distal end surface of the bending section 5, an imaging unit, an opening portion for a channel [a fluid transmitting/receiving conduit] such as a forceps hole, a cleaning nozzle, a suction nozzle, and others are provided. Further, the operating unit 3 includes a grip section 3a gripped by an operator, and the proximal end of the inserting section 4 is connected to the distal end side of the grip section 3a in the operating unit 3 through a member such as a bending stopper.

The endoscope component unit 7 is constituted of a light source apparatus which generates illumination light to be applied to an observation target region, a video processor which executes predetermined image processing to an acquired video signal, a monitor which displays the video signal as an observation image, a keyboard which is an input unit, and others.

Furthermore, a bottle 10 which stores a cleaning liquid used for cleaning or the like is detachably disposed to a support of the trolley 2. Moreover, a non-illustrated air supply pump unit is arranged in the endoscope component unit 7. Additionally, a suction unit 8 which sucks, under normal conditions, a liquid or a gas for cleaning jetted into a lumen from the cleaning nozzle arranged at the distal end of the inserting section, a living fluid including blood, and a removed small piece of a biotissue is provided on a shelf of the trolley 2.

The endoscope main body 1 is connected to the light source unit by using a connector through a universal cord 6. The universal cord 6 includes signal lines through which video signals and others are transmitted, a supply path for a gas and a liquid (an air supply/liquid supply channel) formed of a tube, and a discharge path as well as a light guide formed of an optical fiber. The connector connected to the endoscope component unit 7 side of the universal cord 6 is branched for the signal lines, the tube, and the light guide, and connected to respective constituent portions.

Further, a conduit opening/closing apparatus 11 according to this embodiment is disposed to an opening portion of the operating unit 3 on the distal end side, i.e., a mouthpiece portion of a forceps plug [a fluid flow mouthpiece section 3b] provided between the grip section 3a and the proximal end of the inserting section 4, and it is connected to an external suction pump 21 through a suction tube 22. In this embodiment, as an operation method, it is assumed that the external suction pump 21 is maintained in a driving state (a suction operating state) from start of a treatment to end of the treatment, and a valve switching operation in the conduit opening/closing apparatus 11 is carried out as required so that a suction state and non-suction state are switched.

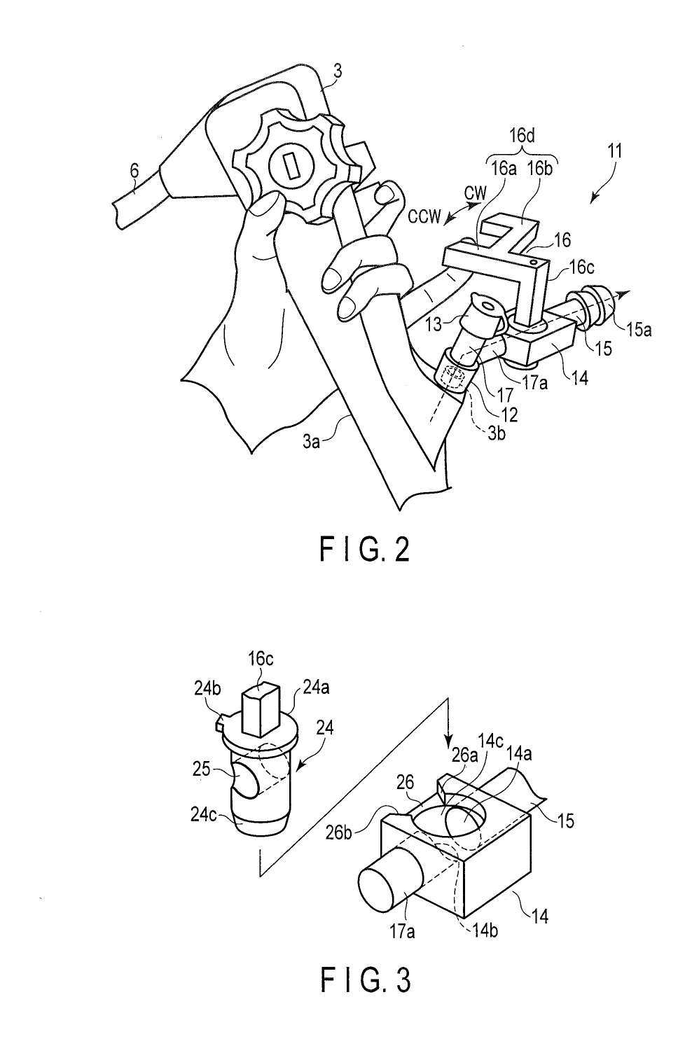

FIG. 2 shows an appearance structural example of the conduit opening/closing apparatus 11 according to the first embodiment. FIG. 3 shows an exploded structural example of a switching section main body 14.

This conduit opening/closing apparatus 11 is constituted of a connecting section 12 detachably disposed to the mouthpiece section 3b of the forceps plug, a T-shaped pipe 17 serving as a main conduit [a first pipe-like section] which is water-tightly coupled with the mouthpiece section by the connecting section 12 at one end and fastened by a forceps plug 13 at the other end, the switching section main body 14 which switches suction operations, a connection pipe 15 to be connected to the suction tube 22, and an operation lever 16 disposed to the switching section main body 14. Although a material of this conduit opening/closing apparatus 11 is not restricted as long as sterilization or cleaning is possible, this apparatus is made of a resin material or a metal material such as stainless or a stainless alloy in this embodiment.

The connecting section 12 is fitted to the mouthpiece section 3b and water-tightly disposed by using packing. Furthermore, a screw thread may be formed on the mouthpiece section 3b to dispose the connecting section 12 by screwing. A flow path piercing through the inside of the switching section main body 14 is formed in the switching section main body 14, a branch pipe 17a of the T-shaped pipe 17 is fixed to an inflow port side 14b, and the connection pipe 15 is fixed to an outflow port side 14c. In this embodiment, the branch pipe 17a of the T-shaped pipe 17, the switching section main body 14, and the connection pipe 15 form a water-tight flow path [a second pipe-like section] from which leakage to the outside does not occur. Mirror polishing or the like may be performed to an inner surface of this flow path to reduce flow path resistance, thereby facilitating sterilization. On a distal end side of the connection pipe 15, a spigot section 15a to which annular convex portions are successively formed is provided to connect the suction tube 22.

As shown in FIG. 3, in the switching section main body 14, a cylindrical hole 14a is formed to divide the inner flow path from above, and a plug section 24 [a switching valve] is turnably fitted in this hole. The plug section 24 has an inner diameter equal to flow path diameters of the branch pipe 17a and the connection pipe 15, a flow path 25 piercing through the plug section 24 is formed, and a bottom portion 24c has a tapered truncated conical shape. A flange portion 24a expanding outward beyond a plug outer periphery is formed at an upper portion of the plug section 24, and a base portion 16c of the operation lever [a fluid switching lever] 16 is erected on an upper surface of the plug section. A protruding portion 24b protruding in a horizontal direction is formed on the flange portion 24a.

The operation lever 16 according to this embodiment is constituted of a main lever section [a first lever section] 16a which is bent at a substantially right angle from the base portion 16c and extends in an L-like shape, and a sub-lever section [a second lever section] 16b which is bifurcated from the main lever section 16a, has an L-like shape bent at a substantially right angle, and has an arm section extending parallel to the main lever section 16a. The main lever section 16a and the sub-lever section 16b constitute a channel-shaped or U-shaped finger abutting section 16d.

FIG. 2 shows a state where a tip of a fifth finger of a hand (here, a left hand is shown) gripping the operating unit 3 enters the finger abutting section 16d, and the finger is hooked on the main lever section 16a. A movable range limiting section 26 to regulate a turning range (a turning angle) of the operation lever 16 fitted in the hole 14a is provided to the switching section main body 14. This movable range limiting section 26 is a step-like notch which allows the protruding portion 24b to function as a stopper and has a fan-like flat surface connected to the hole 14a, and a movable range is limited by limiting walls 26a and 26b on both sides. When the protruding portion 24b abuts on one of the limiting walls 26a and 26b, e.g., the limiting wall 26a, a through flow path is formed by connecting the flow path 25 of the plug section 24 to the flow paths of the T-shaped pipe 17 and the connection pipe 15 (an opened state of the switching valve), thereby achieving a maximum (100%) suction amount. On the other hand, when the protruding portion 24b abuts on the limiting wall 26b, the plug section 24 is rotated, e.g., 90°, and a plug peripheral surface on which flow path 25 is not formed closes the flow paths of the T-shaped pipe 17 and the connection pipe 15 (a closed state of the switching valve) , thereby blocking the flow paths. As regards the limiting wall 26a and the limiting wall 26b, since use in the fully opened state or the fully closed state of the operation lever 16 is basically supposed, for example, the turning angle of the plug section 24 is set to 90° (an axial direction of the through flow path indicated by a dotted line is assumed to be 0°). It is to be noted that, when the protruding portion 24b is present between the limiting wall 26a and the limiting wall 26b, since an aperture of the flow path 25 is increased or decreased in correspondence with an angle at which the plug section 24 turns to the apertures of the flow paths of the T-shaped tube 17 and the connecting section 15, and hence the suction amount can be adjusted.

In this embodiment, when the tip of the fifth finger enters the finger abutting section 16d and sub-lever section 16b is pushed and retreated by the finger in a CW direction to press the protruding portion 24b against the limiting wall 26a, suction is performed. Further, when the main lever section 16a is drawn by the finger and rotated in a CCW direction and the protruding portion 24b is pressed against the limiting wall 22b, the suction is terminated. In this example, the operation lever 16 is set to enter the closed state when it is drawn by the finger since a movement of the finger can easily respond and an operation can be readily performed when emergency is involved. Furthermore, in this embodiment, the main lever section 16a and the sub-lever section 16b have the same arm length, the main lever arm 16a may be formed with a longer length so that the finger can be more easily hooked at the time of closing the switching valve.

In this embodiment, although the U-like or channel-like arm shape is taken as an example, a non-illustrated C-like shape having an arc in a linear portion of an arm section may be adopted.

As described above, in the conduit opening/closing apparatus 11 according to this embodiment, the finger can be stretched to perform the opening/closing operation of the conduit opening/closing apparatus 11, i.e., the switching operation of the conduits while the hand operating the operating unit 3 is in a gripping state, and the suction can be carried out. Thus, it is not necessary to perform the switching operation of the operation lever 16 while releasing the other hand gripping the inserting section 4 like conventional examples, and unwanted movement of an observation viewing field can be avoided. Furthermore, it is not necessary to ask a nearby staff and the like to perform the opening/closing operation of the operation lever, and opening/closing can be carried out by an operator him/herself at desired timing, and the suction amount can be also adjusted on the basis of a degree of opening/closing.

### Second Embodiment

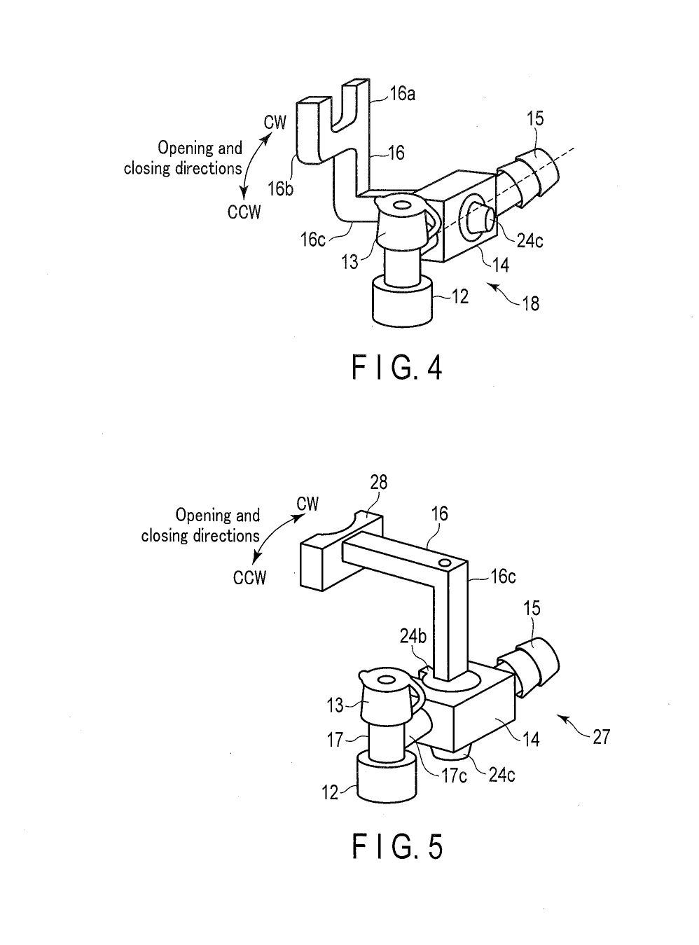

FIG. 4 shows an appearance structural example of a conduit opening/closing apparatus 11 according to a second embodiment.

In a conduit opening/closing apparatus 18 according to this embodiment, constituent portions equal to those shown in FIG. 1 are used, and like reference numerals are given to omit a detailed description.

This is a configuration where positions of a T-shaped tube 17 and a connection tube 15 secured to a switching section main body 14 are counterchanged at the time of manufacture to the configuration shown in FIG. 11. According to this configuration, a turning direction of an operation lever 16 is changed from the horizontal direction to the vertical direction to the conduit opening/closing apparatus 11 according to the first embodiment shown in FIG. 1.

According to this embodiment, a tip of a fifth finger is inserted into a finger abutting section 16d, a main lever section 16a is pushed by the finger and turned in a CW direction, and a protruding portion 24b is pressed against a limiting wall 26a, thereby performing the suction. Moreover, a sub-lever section 16b is drawn by the finger and turned in a CCW direction, and a protruding portion 24b is pressed against a limiting wall 26b, thereby terminating the suction. In this example, likewise, a setting is configured so that the closing state is achieved by drawing the lever with the use of the finger, and an immediate response can be made to facilitate the operation when emergency is involved.

According to this embodiment, the same functions and effects as those in the first embodiment can be provided. Additionally, since a turning direction of the operation lever 16 can be changed to the vertical direction with equivalent constituent portions, operations which suit an operator's taste can be realized.

### Third Embodiment

FIG. 5 shows an appearance structural example of a conduit opening/closing apparatus 11 according to the third embodiment.

In a conduit opening/closing apparatus 27 according to this embodiment, like reference numerals denote constituent portions equal to the constituent portions shown in FIG. 1, and different constituent portions which are technical characteristics will be described in detail.

In this embodiment, an operation lever 16 is constituted of an L-shaped main lever section 16a and an abutting section 28 which is fixed to a distal end portion of the main lever section 16a. A turning direction of the operation lever 16 is equal to that of the operation lever 16 according to the first embodiment, and the operation lever 16 is turned in a substantially horizontal direction. As a matter of course, like the second embodiment, the turning direction of the operation lever 16 can be changed by counterchanging positions of a T-shaped tube 17 and a connection tube 15 fixed to a switching section main body 14 at the time of manufacture.

The abutting section 18 has a prismatic shape, and its surface on which a finger abuts is formed into a cylindrical concave surface. A tip of the finger is put on this concave surface, the concave surface is pressed by the finger and turned in a CW direction, and a protruding portion 24b is pressed against a limiting wall 26a, thereby performing the suction. Alternatively, the finger is pressed against the concave surface of the abutting section 28, the concave surface is turned in a CCW direction to be drawn toward an operator's side, and the protruding portion 24b is pressed against a limiting wall 26b, thereby terminating the suction. A depth of the concave surface of the abutting section 28 is a design matter, and it may be selected on the basis of the operator's convenience at the time of manufacture assembling. For example, the abutting section 28 and a disposing position of the abutting section 28 may be fabricated on the basis of a fixing system using screwing of a screw structure, and the abutting sections 28 having the concave surfaces with different depths formed thereto may be prepared in advance. Furthermore, many small protrusions, grooves, and the like may be formed on the concave surface of the abutting section 28, and anti-slip finishing may be applied.

As described above, according to this embodiment, since the concave surface of the abutting section 28 has the shallow depth, the tip of the finger can instantaneously and easily abut on and move away from the concave surface as compared with the U-shaped operation lever, and the opening/closing operation with a quick response can be realized. Moreover, in this embodiment, the same functions and effects as those in the first embodiment can be exerted.

### Fourth Embodiment

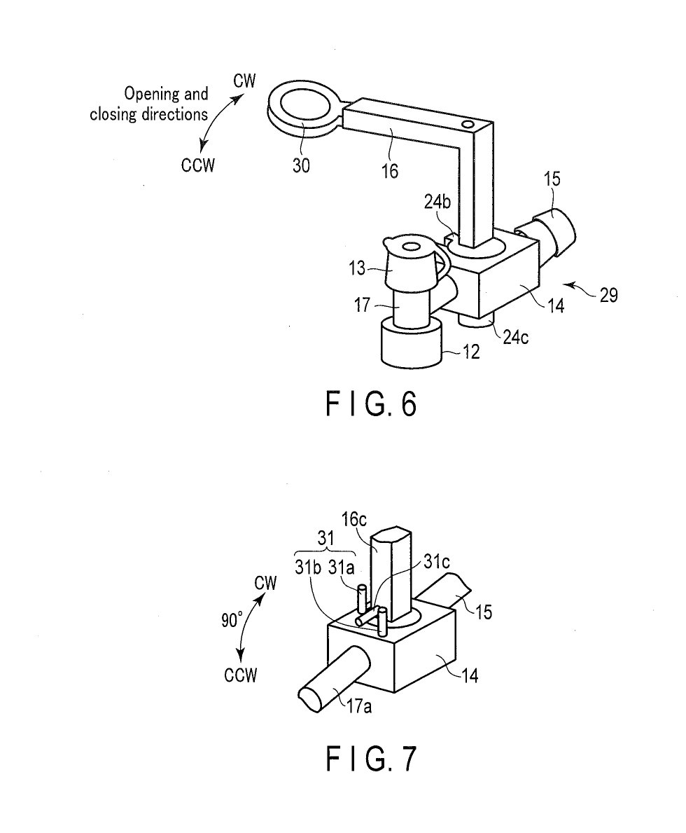

FIG. 6 shows an appearance structural example of a conduit opening/closing apparatus according to a fourth embodiment.

In the conduit opening/closing apparatus 29 according to this embodiment, like reference numerals denote constituent portions equal to the constituent portions shown in FIG. 1, and different constituent portions which are technical characteristics will be described in detail.

In this embodiment, an operation lever 16 is constituted of an L-shaped main lever section 16a and a finger hook section 30 which is fixed to a distal end portion of the main lever section 16a.

A turning direction of the operation lever 16 is equal to that of the operation lever 16 according to the first embodiment, and the operation lever 16 is turned in a substantially horizontal direction. As a matter of course, the turning direction of the operation lever 16 can be changed by counterchanging positions of a T-shaped pipe 17 and a connection pipe 15 fixed to a switching section main body 14 at the time of manufacture like the second embodiment.

The finger hook section 30 has a ring-like shape, and a tip of a finger is inserted into a ring to perform an operation.

First, a tip of a finger (e.g., a fifth finger) of a hand gripping an operating unit 3 is inserted into the ring of the finger hook section 30, and the finger hook section 30 is pushed out and turned in a CW direction by the finger to press a protruding portion 24b against a limiting wall 26a, thereby performing the suction. Alternatively, the finger is inserted into the finger hook section 30, and the finger hook section 30 is turned in a CCW direction toward an operator's side to press the protruding portion 24b against a limiting wall 26b, thereby terminating the suction.

Although FIG. 6 shows the ring shape which is a true circle and has a flat surface, this shape is not restricted, and it may be an elliptic shape with an arbitrary outer shape (a frame shape), and its cross section may have a circular shape or an elliptic shape without being restricted to a rectangular shape alone. As regards an inner diameter of the ring, finger hook sections 30 having different inner diameters may be previously prepared at the time of manufacture so that a diameter which is suitable for the operator's finger can be selected. Further, although FIG. 6 shows that a ring surface of the finger hook section 30 is parallel to the turning direction of the operation lever 16, the ring surface may be inclined to facilitate insertion of the operator's finger.

As described above, according to this embodiment, since the finger hook section 30 has a ring shape, the hooked fingertip is hardly disengaged from the link section, and an operation can be assuredly performed. Furthermore, in this embodiment, the same functions and effects as those in the first embodiment can be carried out.

### Fifth Embodiment

FIG. 7 shows an appearance structural example of a movable range limiting section 31 of a conduit opening/closing apparatus 29 according to a fifth embodiment.

In the movable range limiting section 31 according to this embodiment, like reference numerals denote constituent portions equal to the constituent portions shown in FIG. 1, and different constituent portions which are technical characteristics will be described in detail.

The movable range limiting section 31 is constituted of fixing pins 31a and 31b which define a turning range, and a stopper pin 31c. An operation lever 16 is assumed to be used in a fully opened state or a fully closed state using a plug section 24 which are realized by the fixing pins 31a and 31b.

Thus, like the movable range limiting section according to the first embodiment, since the plug section 24 rotates, e.g., 90° and a flow path 25 connects or blocks flow paths of a T-shaped tube 17 and a connection tube 15, the fixing pins 31a and 31b are set in a range of 0° to 90° as a turning angle of the plug section 24. It is to be noted that, when the stopper pin 31c is present between the fixing pin 31a and the fixing pin 31b, since an aperture of the flow path 25 increases or decreases to apertures of the flow paths of the T-shaped pipe 17 and the connection pipe 15 in correspondence with the turning angle of the plug section 24, a suction amount can be adjusted.

According to this embodiment, the movable range limiting section 32 can be configured with a simple structure.

### Sixth Embodiment

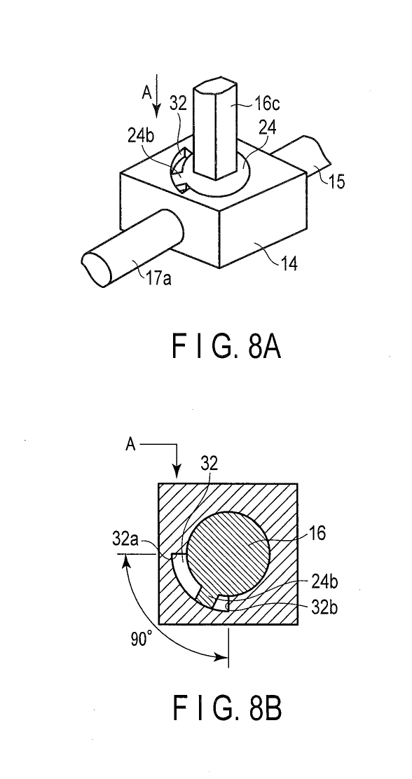

FIG. 8A is a view showing an appearance structural example of a movable range limiting section of a conduit opening/closing apparatus according to a sixth embodiment, and FIG. 8E is a view showing a cross-sectional structure showing the conduit opening/closing apparatus from above.

In the movable range limiting section 32 according to this embodiment, like reference numerals denote constituent portions equal to the constituent portions shown in FIG. 1, and different constituent portions which are technical characteristics will be described in detail.

The movable range limiting section 32 is constituted of a groove having a fan-like flat surface on a switching section main body 14, and a protruding portion 24b of a plug section 24 arranged in the groove. This groove has the fan-like flat surface connected to a hole 14a, and a movable range of the protruding portion 24b is limited by limiting walls 32a and 32b on both sides.

Like the first embodiment, for example, when the protruding portion 24b abuts on the limiting wall 32a, the movable range limiting section 32 connects a flow path 25 of the plug section 24 with flow paths of a T-shaped pipe 17 and a connection pipe 15 to form a through flow path, thereby realizing a maximum suction amount. Moreover, the plug section 24 turns when it abuts on the limiting wall 32b, and the flow paths of the T-shaped pipe 17 and the connection pipe 15 are closed with a plug peripheral surface having no flow path 25 formed thereon, thus blocking the flow path.

According to this embodiment, the same functions and effects as those in the first embodiment are exerted.

Additionally, a flat structure having the same height as an upper surface of a switching section main body 14 can be provided.

According to the present invention it is possible to provide the conduit opening/closing apparatus which enables an operator to easily switch conduits him/herself, and the insertion system including the conduit opening/closing apparatus.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A conduit opening/closing apparatus comprising:
a connecting section which is detachably connected to a fluid flow mouthpiece provided between a grip section to be griped, which is provided on a proximal end side of an inserting section which has a distal end and a proximal end and whose distal end side is inserted into a lumen, and the proximal end of the inserting section;
a first pipe-like section which is consecutively connected to the connecting section and provided along an axial direction of the fluid flow mouthpiece;
a second pipe-like section which is connected to the first pipe-like section, has a flow path which extends in a direction crossing an axial direction of the first pipe-like section and in which a fluid having passed through the fluid flow mouthpiece flows;
a switching valve which is provided in the middle of the flow path, and switches a passing state and a non-passing state of the fluid by connecting or interrupting the flow path on the basis of a turning motion; and
an operation lever which is erected on the switching valve, turns the switching valve, and is disposed to be placed on the grip section side of the fluid flow mouthpiece.

2. The apparatus according to claim 1, comprising:
a finger abutting section comprising a bifurcated structure in which a first receiving section which is extended on a distal end side of the operation lever and receives external force to turn the operation lever in a clockwise direction faces a second receiving section which is branched from the operation lever and receives external force to turn the operation lever in a counterclockwise direction.

3. The apparatus according to claim 1, comprising:
a finger abutting section which is provided on a distal end side of the operation lever, formed into a shape comprising either a ring or a concave surface, and comprises a third receiving section which receives external force to turn the switching valve.

4. The apparatus according to claim 3,
wherein the operation lever is formed into an L-like shape, and comprises a pillar-like first lever section erected and extended from the switching valve, and a second lever section bent and extended from the first lever section, and the finger abutting section is provided on a distal end side of the second lever section.

5. The apparatus according to claim 1,
wherein the switching valve further comprises a turning angle limiting section which defines an angle range in which the operation lever turns, and switches between a state where the flow path is connected and a state where the flow path is blocked.

6. An insertion system comprising:
an inserting section which has a distal end and a proximal end, and whose distal end side is inserted into a lumen;
a grip section which is provided on the proximal end side of the inserting section, and gripped by an operator;
a fluid flow mouthpiece which is provided between the proximal end of the inserting section and the grip section;
a fluid transmitting/receiving conduit which is consecutively connected from an opening portion provided at the distal end of the inserting section to the fluid flow mouthpiece; and
the conduit opening/closing apparatus according to claim 1 which is connected to the fluid flow mouthpiece.
